(19) United States
(12) Patent Application Publication
(10) Pub. No.: US 2026/0130618 A1
(43) Pub. Date: May 14, 2026

(54) PORTABLE ELECTROCARDIOGRAM DEVICE

(71) Applicant: USTATION INC., Chuncheon-si, Gangwon-do (KR)

(72) Inventor: Jun Ho BAEK, Chuncheon-si, Gangwon-do (KR)

(21) Appl. No.: 19/118,751

(22) PCT Filed: Nov. 8, 2022
(86) PCT No.: PCT/KR2022/018956

§ 371 Date: Apr. 4, 2025

(30) Foreign Application Priority Data
Oct. 7, 2022 (KR) 10-2022-0128444

(51) Int. Cl. A61B 5/332; A61B 5/26; A61B 5/28; A61B 5/308; A61B 5/339; A61B 5/352

(52) U.S. Cl. CPC A61B 5/332; A61B 5/26; A61B 5/28; A61B 5/308; A61B 5/339; A61B 5/352; A61B 2560/0431

(57) ABSTRACT

Proposed in the present invention is a portable electrocardiogram device that excludes, from an electrocardiogram graph, signals that are determined to be noise to prevent a user from being alarmed by unnecessary noise, and distinguishes invalid signals from electrode signals to provide the user with accurate electrocardiogram signals and graphs. To this end, the present invention may comprise: a sensor unit; a display device; and a control unit.

### TECHNICAL FIELD

The present disclosure relates to a portable electrocardiogram device, and more particularly, to a portable electrocardiogram device capable of minimizing vibration and noise generated in the portable electrocardiogram device to thereby generate a precise electrocardiogram signal and graph.

### BACKGROUND ART

An electrocardiogram is a representation of action currents and action potential differences according to heart contractions in a form of a curve, and is very important in diagnosing a condition of a heart. An electrocardiogram is generally measured using expensive electrocardiogram devices in hospitals. In addition, portable electrocardiogram devices are also widely used by patients to diagnose daily conditions.

Since an electrocardiogram device measures action currents according to heart contractions, a measured frequency band corresponds to low frequencies of several tens of hertz or less. Such a level of low frequencies corresponds to a frequency band similar to a low frequency generated when a human body moves, a low frequency generated when breathing, and a low frequency generated when other muscles in the human body move.

Therefore, when an electrocardiogram is measured with the electrocardiogram device, a subject needs to be measured in a stable environment as possible and be careful to minimize body movement to prevent low-frequency noise caused by a human body.

However, in such a case that an electrocardiogram is measured using a portable electrocardiogram device,
1) when the portable electrocardiogram device is held in a hand and operated,
2) when coughing occurs or a body is temporarily moved during the measurement, and
3) when an electronic device that generates low-frequency noise is present in a vicinity, a correct electrocardiogram graph is not obtained by the measurement.

Items 1) and 2) describe situations that may occur to anyone when a portable electrocardiogram device is held in a hand and operated and are not easily controlled unless assisted by a nurse or a third party. Item 3) describes a situation that may not be easily recognized by a user, and in this case, a method of performing filtering by a portable electrocardiogram device is most effective.

The situation in Item 3) may be suppressed by performing filtering using a portable electrocardiogram device. However, with respect to Items 1) and 2), filtering is not easily performed, and thus, these situations are reflected in an electrocardiogram graph and cause unnecessary noise in electrocardiogram measurement on a user, thus alarming the user or resulting in unnecessary misunderstandings.

Korea Patent Publication No. 10-2021-0080866 (Electrocardiogram measurement device and reading algorithm using low-power long-distance communication network)

Korean Patent Registration No. 10-1555569 (Method for detecting electrocardiogram signal, method for displaying electrocardiogram signal, and electrocardiogram signal detecting apparatus)

### DISCLOSURE OF INVENTION

### Technical Problem

Therefore, the present disclosure has been made in view of the above problems, and it is an object of the present disclosure to provide a portable electrocardiogram device that minimizes noise generated when an electrocardiogram is measured by holding the portable electrocardiogram device in a hand.

Another object of the present disclosure is to provide a portable electrocardiogram device capable of removing atypical signals generated from electrode signals when an electrocardiogram is measured, and providing a user with a correct electrocardiogram signal reconstructed using only typical signals.

### Technical Solution

To accomplish the above object, according to one aspect of the present disclosure, there is provided a sensor unit configured to detect an electrocardiogram using an electrode in contact with a finger of a user; a display device configured to display electrocardiogram information; and a control unit configured to: acquire electrode signals through the sensor unit, detect a plurality of R signals from the electrode signals, and generate a normalizing signal obtained by rearranging cycles of the R signals; and extract a signal group belonging to a normal category on a basis of an integral value of each unit signal constituting the normalizing signal, and on a basis of the extracted signal group, construct an electrocardiogram graph to display the electrocardiogram graph on the display device.

Here, the normalizing signal may be a signal obtained by rearranging the electrode signals on a basis of an average cycle of in R signals of a plurality of signals from a time point of starting measurement when the sensor unit measures the electrode signals.

Here, when an integral value of an electrode signal including one section R, among the electrode signals including the plurality of sections R, is differentiated from an average value in sections R of other electrode signals, the control unit may desirably normalize the electrode signal including the one section R with the differentiated integral value to correspond to the average value in the sections R.

Desirably, a gyro sensor may be further included, and the control unit may block measurement of the electrode signals when measurement values for three axes of the gyro sensor deviate from a preset reference value.

At this time. when one of the measurement values for the three axes of the gyro sensor is within the reference value, the control unit may measure the electrode signals and generate the electrocardiogram graph based on the received electrode signals.

Here, in a case of being within the reference value for preset reference time, the control unit may desirably generate the electrocardiogram graph.

Here, the control unit may extract the signal group belonging to the normal category through the normalizing signal, de-normalize the signal group to return to an original form of the electrode signals, and then, construct the electrocardiogram graph.

### Advantageous Effects

According to the present disclosure, noise generated when measurement is performed by holding a portable electrocardiogram device in a hand may be minimized.

Signals determined as noise may be excluded from an electrocardiogram graph to prevent a user from being alarmed by unnecessary noise.

In addition, by removing invalid atypical signals from electrode signals, an accurate electrocardiogram graph may be provided to a user.

### BRIEF DESCRIPTION OF THE DRAWINGS

FIG. 1 illustrates a reference diagram of a waveform of an electrocardiogram signal.

FIG. 2 illustrates a conceptual block diagram of a portable electrocardiogram device according to one embodiment of the present disclosure.

FIG. 3 illustrates a reference diagram showing a method of performing normalizing.

FIG. 4 illustrates a conceptual diagram showing a method of adjusting intervals of electrode signals.

FIG. 5 illustrates a product image of the portable electrocardiogram device according to one embodiment of the present disclosure.

FIG. 6 illustrates a menu screen of the portable electrocardiogram device according to one embodiment of the present disclosure.

FIG. 7 illustrates an example of displaying an electrocardiogram graph for a user in correspondence with a graph menu selected by the user from the menu screen.

### MODE FOR CARRYING OUT THE INVENTION

A PQRST wave mentioned with respect to an electrocardiogram signal in the present disclosure refers to a waveform presented in a graph to show an electrical activity that occurs while a heart contracts once.

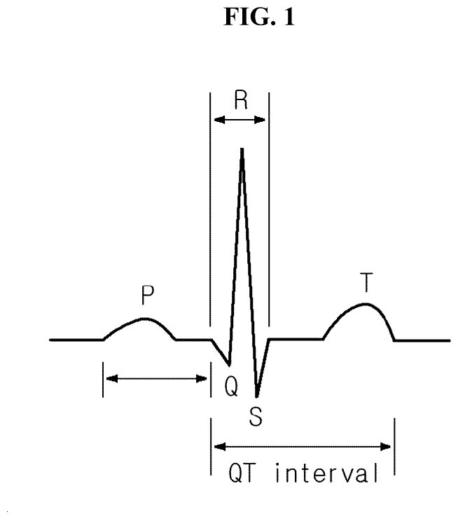

In FIG. 1, a P wave refers to a waveform that starts when an atrium contracts, and occurs for 0.05 to 0.1 seconds.

In FIG. 1, a QRS wave is a waveform that occurs during time when a ventricle contracts and represents a maximum peak in an electrocardiogram signal, and a time period during which the maximum peak continues is referred to as a “section R.”

In FIG. 1, points positioned on left and right sides of an R waveform to represent minus (−) values from an average value are denoted as Q and S, respectively.

In FIG. 1, an electrical signal that occurs during a rest period of a ventricle after the ventricle has completed one contraction is referred to as a T wave.

In FIG. 1, a time period from a start to an end of one contraction of the ventricle is referred to as a QT interval.

An atypical signal described in the present disclosure may refer to a signal generated by external noise. This may correspond to a signal generated when a user moves during electrocardiogram measurement or shakes the portable electrocardiogram device according to the present disclosure.

A typical signal described in the present disclosure may refer to a correct electrocardiogram signal measured with respect to a user.

Hereinafter, the present disclosure will be described in detail with reference to the accompanying drawings.

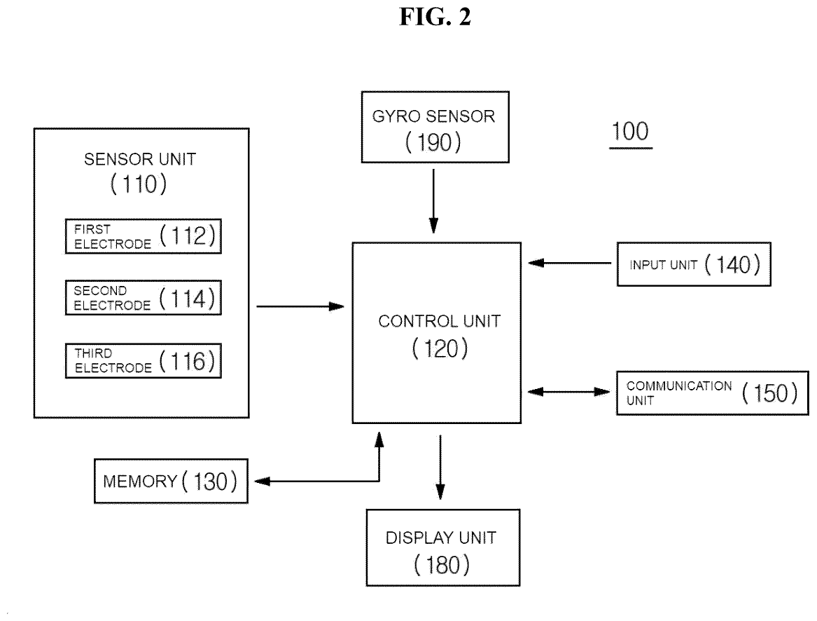

FIG. 2 illustrates a conceptual block diagram of a portable electrocardiogram device according to one embodiment of the present disclosure.

A portable electrocardiogram device 100 according to one embodiment of the present disclosure may be configured to include a sensor unit 110, a control unit 120, a memory 130, an input unit 140, a communication unit 150, a display unit 180, and a gyro sensor 190.

The sensor unit 110 includes three electrodes 112, 114, and 116 in contact with three portions of fingers of a user and detects electrode signals.

Here, signals formed as signals of the three electrodes corresponds to electrocardiogram signals. Therefore, an electrode signal described in the present disclosure may often be used interchangeably with a term, electrocardiogram signal in terms of expressions and meanings.

The sensor unit 110 detects electrode signals corresponding to action potentials that occur along a neurotransmission pathway of a heart through each of the included electrodes 112, 114, and 116 and transmits the electrode signals to the control unit 120.

Here, the sensor unit 110 may include the electrodes 112 and 114 on one side surface of an outer circumference of the portable electrocardiogram device 100 to be in contact with two fingers of a left arm, respectively, and the electrode 116 on another side surface of the outer circumference of the portable electrocardiogram device 100 to be in contact with one finger of a right arm.

Each of the electrodes 112, 114, and 116 may be desirably made of a material with high electrical conductivity.

Additionally, the electrodes 112, 114, and 116 may be configured as dry electrodes. When the electrodes 112, 114, and 116 are implemented as dry electrodes and an electrocardiogram of a user is to be measured, the electrocardiogram may be measured using the portable electrocardiogram device 100 according to the present embodiment without having to use a separate gel.

The control unit 120 minimizes external electromagnetic noise by performing low pass filtering on a signal detected by the sensor unit 110 and performs analog-to-digital conversion to construct an electrocardiogram (ECG) signal.

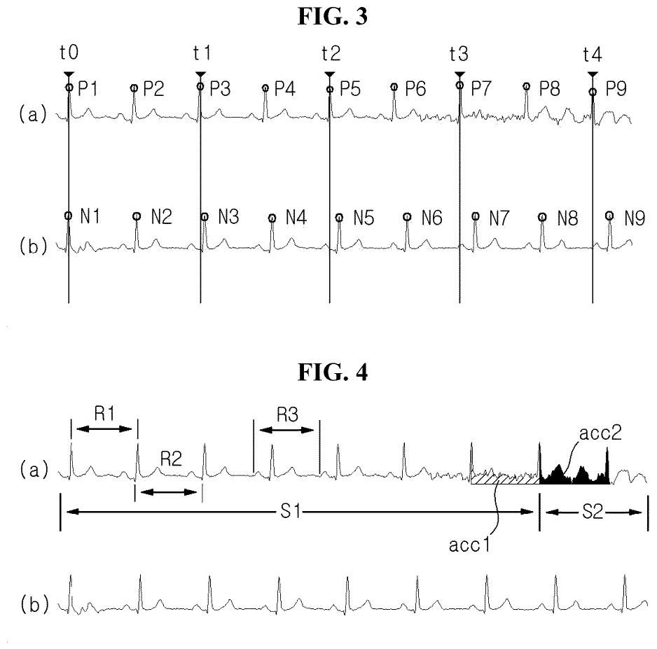

The control unit 120 may normalize an electrode signal measured by the sensor unit 110, and then, de-normalize the normalized electrode signal to remove atypical signals and extract a group of typical signals to regenerate an electrocardiogram graph. This will be explained with reference to FIGS. 3 and 4.

FIG. 3 illustrates a reference diagram showing a method of performing normalizing.

Referring to FIG. 3 together, (a) of FIG. 3 illustrates an example of indicating intervals of electrode signals using t0, t1, t2, t3, and t4 as reference lines with respect to sections R included in the electrode signals. In addition, (b) of FIG. 3 shows electrode signals continuously measured by a same user as that in (a) of FIG. 3.

Referring to (a) of FIG. 3 and (b) of FIG. 3, it may be seen that the electrode signals do not have uniform intervals when the same user measures an electrocardiogram using the portable electrocardiogram device 100 according to an embodiment.

In (a) of FIG. 3, electrocardiogram signals are indicated as P1 to P9 with reference to peak values in the sections R. In (b) of FIG. 3, electrocardiogram signals are indicated as N1 to N9 with reference to peak values in the sections R.

Intervals of electrode signals continuously measured by one user using one portable electrocardiogram device 100 are not identical to each other as shown in FIG. 3.

Referring to t1 to t4 indicated as the reference lines to compare positions of P1 to P9 with those of N1 to N9, it may be seen that although starting points of P1 and N1 are identical, the positions of P9 and N9 are not identical and N9 is indicated later than the reference line t4.

With reference to distances in two to five sections R, the control unit 120 calculates an average value between the sections R, and equalizes subsequent intervals of the electrode signals based on the calculated average value in the sections R. This corresponds to normalizing described in the present disclosure.

The control unit 120 may increase or decrease signal widths of electrode signals input thereafter based on the average value in the sections R in correspondence with a same time interval to match the average value in the sections R, and calculate an integral value for a signal in each cycle with respect to the electrode signals normalized by the average value in the sections R.

Then, the normalized electrode signals are integrated for each cycle of the electrode signals by the control unit 120 and used to distinguish between typical and atypical signals.

In addition, the control unit 120 removes the atypical signals, then, reconstructs electrode signals using only the typical signals, and performs de-normalizing after the reconstructing of the electrode signals using the typical signals.

The control unit 120 restores the normalized electrode signals to an original time interval through the de-normalizing. When the de-normalizing is performed, the sections R for the respective electrode signals do not have a uniform time interval, and an original form of signals measured from the respective electrodes 112, 114, and 116 by the sensor unit 110 are restored.

Then, the control unit 120 may output, to the display unit 180, electrode signals from which the atypical signals have been removed through the de-normalizing.

The control unit 120 may process electrode signals in an order according to paragraphs presented below.
1) A plurality of sections R are detected from electrode signals,
2) normalizing is performed to have a constant interval with reference to peak values of the detected sections R to thereby generate normalizing signals,
3) each of the normalizing signals is integrated in units of one cycle to determine atypical signals having an integral value greater or less than an average value of other integral values by 30% to 70% or more,
4) a group of remaining normalizing signals other than the atypical signals is extracted, and
5) an electrocardiogram graph is created using the extracted group of normalizing signals.

Here, a method of removing atypical signals and reconstructing an electrocardiogram signal (an electrode signal) using only typical signals are described with reference to FIG. 4 together.

FIG. 4 illustrates a conceptual diagram showing a method of adjusting intervals of electrode signals.

Referring to FIG. 4, it may be seen that sections R are arranged at relatively uniform intervals in sections S1 and S2 in (a) of FIG. 4, but the section S2 corresponds to an atypical section in which an integral value acc2 for signals in the section S2 is higher than an average integral value acc1 in the section S1.

A reason for which the integral value acc2 in the section S2 is 30% to 70% higher than the average integral value acc1 in the section S1 is that vibration has occurred in the portable electrocardiogram device 100 or body muscles of a user have moved in the section S2.

Even when the user properly holds the portable electrocardiogram device 100 according to the embodiment and does not apply vibration to the portable electrocardiogram device 100, an atypical signal shown in the section S2 may be generated by current generated when other body muscles of the user move.

Since an atypical signal does not correctly represent an electrocardiogram state of the portable electrocardiogram device 100 according to the embodiment, it is desirable to remove the atypical signal when an electrocardiogram graph is generated by the control unit 120.

At this time, as shown in (a) of FIG. 4, the control unit 120 may be configured to:
integrate a region between peak values in the sections R of the electrode signals (R1), or
integrate a region between neighboring Q waveforms of the electrode signals (R2), or
integrate a region between P waveforms of the electrode signals (R3) to calculate integral values for respective electrode signals in units of one cycle of the electrode signals (electrocardiogram signals).

The calculated integral values may be cumulatively integrated to obtain an average value.

For example, when it is assumed that integral values of a first electrode signal, a second electrode signal, a third electrode signal, and a fourth electrode signal among the electrode signals shown in the section S1 are A, B, C, and D, respectively, the control unit 120 calculates an average value as (A+B)/2 when the second electrode signal is measured.

When the third electrode signal is input to the control unit 120, the control unit 120 calculates an average value as (A+B+C)/3.

When the fourth electrode signal is input to the control unit 120, the control unit 120 calculates an average value as (A+B+C+D)/4.

That is, the control unit 120 sequentially integrates electrode signals which are being input, accumulatively adds integral values for previous electrode signals, and then, divides a result of the accumulative addition by a number of the input electrode signals to calculate an average integral value for the electrode signals.

Since the accumulated integral values are calculated each time a new electrode signal is input to the control unit 120, a computational overload is not imposed on the control unit 120.

The control unit 120 uses an accumulatively calculated average integral value, and when an integral value of a newly input electrode signal is greater or less than the average integral value by 30% to 70% or more, the control unit 120 determines the input electrode signal as an atypical signal, removes the atypical signal, and then, generates an electrocardiogram graph.

For example, since a region S2 in (a) of FIG. 4 corresponds to an atypical signal, the control unit 120 may remove the region S2 and attach an electrode signal input thereafter to a portion from which the region S2 has been removed to generate an electrocardiogram graph in a form as shown in (b) of FIG. 3.

The control unit 120 may detect a button input by the user through the input unit 140 and display a corresponding menu on the display unit 180.

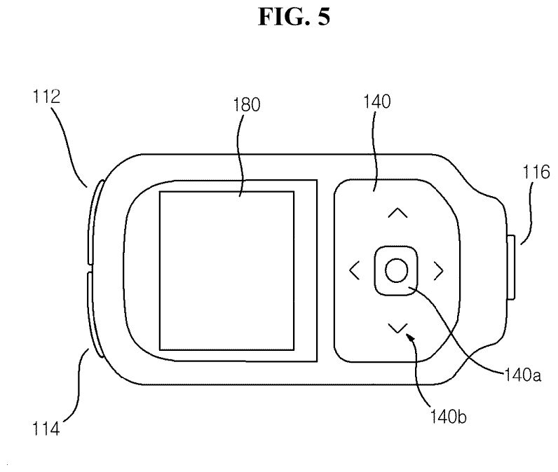

This will be described with reference to FIGS. 5 to 7 together.

First, FIG. 5 illustrates a product image of the portable electrocardiogram device according to one embodiment of the present disclosure.

The portable electrocardiogram device shown in FIG. 5 includes a housing 10, and the display unit 180, the first electrode 112, the second electrode 114, and the third electrode 116 arranged to be exposed on the housing 10, and the input unit 140 arranged to neighbor the display unit 180.

The first electrode 112 and the second electrode 114 are arranged to neighbor each other to be gripped by one hand, and the third electrode 116 is arranged to be spaced apart to be gripped by an opposite hand.

The input unit 140 displayed on a right side in FIG. 5 is provided for the user to call a desired menu and manipulate the called menu. The input unit 140 includes a confirmation button 140a in a center, a direction key button 140b arranged to neighbor the confirmation button 140a. The direction key button 140b corresponds to buttons corresponding to left, right, up, and down.

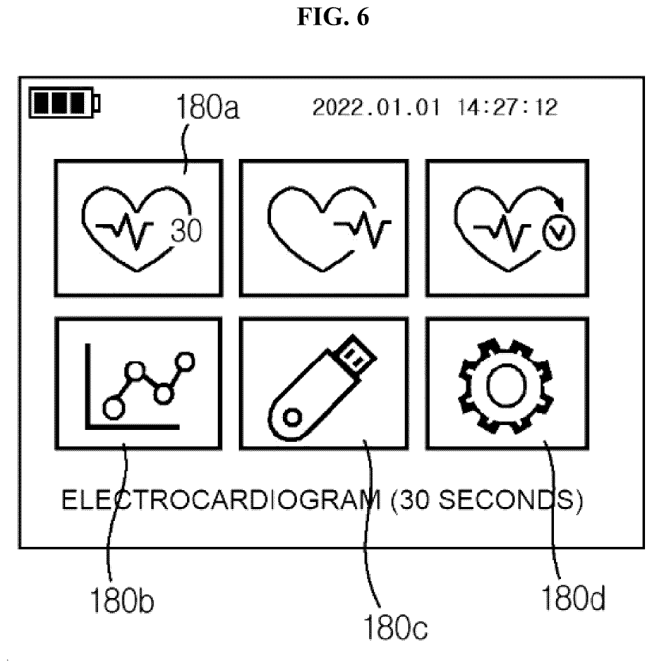

Then, FIG. 6 illustrates a menu screen of the portable electrocardiogram device according to one embodiment of the present disclosure.

The menu screen illustrated in FIG. 6 is a menu screen displayed on the display unit 180 when the portable electrocardiogram device 100 according to an embodiment is turned on, and shows a measurement menu 180a for starting electrocardiogram measurement, a graph menu 180b for displaying an electrocardiogram graph, an external storage menu 180c for transmitting an electrocardiogram signal to an external storage medium (e.g., a universal serial bus (USB)), and an environment setting menu 180d.

The display unit 180 is configured as a touch screen that responds to a touch input. When a user touches a desired menu on the menu screen illustrated in FIG. 6, the control unit 120 responds to this by performing a function corresponding to the menu desired by the user.

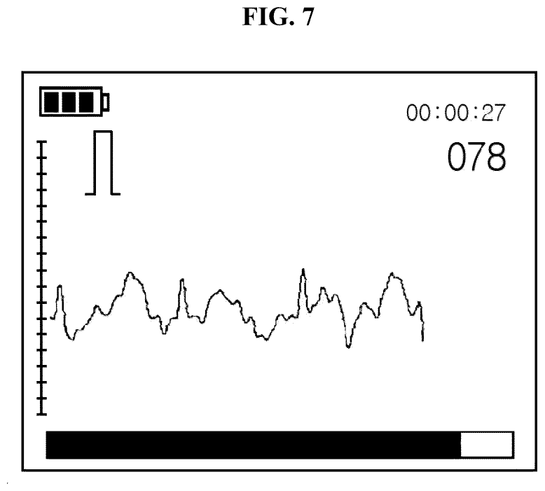

When the user selects the graph menu 180b on the menu screen illustrated in FIG. 6, an electrocardiogram graph as illustrated in FIG. 7 may be displayed on the display unit 180.

FIG. 7 illustrates an example of displaying an electrocardiogram graph for a user in correspondence with the graph menu 180b selected by the user from the menu screen. The electrocardiogram graph shown in FIG. 7 represents a result of reconstruction using typical signals other than atypical signals.

The electrocardiogram graph illustrated in FIG. 7 shows a representation of only typical signals when atypical signals are removed, the atypical signals being generated by shaking or contraction and relaxation of muscles other than a heart which occur when a user grips the portable electrocardiogram device 100 according to an embodiment.

The memory 130 loads an application and an operating system of the control unit 120. When the portable electrocardiogram device 100 according to an embodiment is booted, the operating system may be driven and a temporary storage space needed for the control unit 120 to run the application may be provided.

Here, the application may refer to an application that measures an electrocardiogram signal or generates an electrocardiogram graph, or refer to an application for setting an environment or an application that displays an image or text on the display unit 180.

The communication unit 150 performs data communication with an external computing device through a micro 5-pin connector, a C-type connector, and a USB connector, and may transmit electrocardiogram signals or electrocardiogram graph data to the external computing device.

The external computing device may be any of a desktop computer, a laptop computer, a smartphone, or a server connected to a network, but may correspond to various devices equipped with a central processing unit (CPU), a random access memory (RAM), an operating system or application storage device such as a solid-state drive (SSD), or input/output devices.

The communication unit 150 may have a Bluetooth or Wi-Fi communication function to perform data communication with a smartphone. In this case, the communication unit 150 may wirelessly transmit electrocardiogram signals or electrocardiogram graph information measured on the user.

The gyro sensor 190 detects three-axis shaking of the portable electrocardiogram device 100 according to an embodiment and notifies the control unit 120 of the detected three-axis shaking.

The gyro sensor 190 may detect shaking along an X-axis, a Y-axis, and a Z-axis.
The gyro sensor 190 sets an initial value with reference to a time point at which an electrode signal is measured by the sensor unit 110, and measures a degree of shaking of the portable electrocardiogram device 100 compared to the initial value from the time point of the measurement.
The initial value of the gyro sensor 190 corresponds to a value with respect to the X-axis, Y-axis, and Z-axis at a time point of starting electrocardiogram measurement.

Therefore, the control unit 120 sets, as an initial value, a measurement value with respect to the X-axis, the Y-axis, and the Z-axis of the gyro sensor 190 at a time point of measuring an electrocardiogram signal.
The initial value is not a predetermined value, but represents a sensor value of the gyro sensor 190 at a time point of measuring an electrocardiogram signal.

When the gyro sensor 190 measures a value (a reference value) 20% to 30% greater than the initial value, the control unit 120 regards this as occurrence of an atypical signal, blocks an input of an electrode signal, and stops measuring the electrode signal.

When the value measured by the gyro sensor 190 is within the reference value, the control unit 120 continues to measure an electrode signal.

The present embodiment and drawings attached to the present specification are merely intended to clearly illustrate some of technical ideas included in the present disclosure, and it is deemed obvious that various modifications and particular embodiments that may be easily derived by those skilled in the art within the scope of the technical ideas included in the specification and drawings of the present disclosure are all included in the scope of the present disclosure.

1. A portable electrocardiogram device comprising:
a sensor unit configured to detect an electrocardiogram using an electrode in contact with a finger of a user;
a display device configured to display electrocardiogram information; and
a control unit configured to: acquire electrode signals through the sensor unit, detect a plurality of R signals from the electrode signals, and generate a normalizing signal obtained by rearranging cycles of the R signals; and
extract a signal group belonging to a normal category on a basis of an integral value of each unit signal constituting the normalizing signal, and on a basis of the extracted signal group, construct an electrocardiogram graph to display the electrocardiogram graph on the display device.

2. The portable electrocardiogram device of claim 1, wherein the normalizing signal is a signal obtained by rearranging the electrode signals on a basis of an average value in sections R of a plurality of electrode signals from a time point of starting measurement when the sensor unit measures the electrode signals.

3. The portable electrocardiogram device of claim 2, wherein, when an integral value of an electrode signal comprising one section R, among the electrode signals comprising the plurality of sections R, is differentiated from an average value in sections R of other electrode signals, the control unit normalizes the electrode signal comprising the one section R with the differentiated integral value to correspond to the average value in the sections R.

4. The portable electrocardiogram device of claim 1, further comprising a gyro sensor,
wherein the control unit blocks measurement of the electrode signals when measurement values for three axes of the gyro sensor deviate from a preset reference value.

5. The portable electrocardiogram device of claim 4, wherein, when one of the measurement values for the three axes of the gyro sensor is within the reference value, the control unit receives the electrode signals and generates the electrocardiogram graph based on the received electrode signals.

6. The portable electrocardiogram device of claim 4, wherein, in a case of being within the reference value for preset reference time, the control unit generates the electrocardiogram graph.

7. The portable electrocardiogram device of claim 1, wherein the control unit is configured to:
extract the signal group belonging to the normal category through the normalizing signal; and
de-normalize the signal group to return to an original form of the electrode signals, and then, construct the electrocardiogram graph.
